(19) United States
(12) Patent Application Publication
(10) Pub. No.: US 2009/0012146 A1
(43) Pub. Date: Jan. 8, 2009

(54) SOLUBILITY-ENHANCED PHARMACEUTICAL COMPOSITIONS COMPRISING ZAFIRLUKAST

(76) Inventors: Giridhar Reddy Buggana, Kurnool (IN); Srikanth Basety, Hyderabad (IN); Rahul Sudhakar Gawande, Nagpur (IN); Ravinder Kodipyaka, Kaghaznagar (IN); Srinivas Irukulla, Hyderabad (IN); Indu Bhushan, Hyderabad (IN); Mailatur Sivaraman Mohan, Hyderabad (IN); Praveen Kumar Bembaloor, Coorg (IN)

Correspondence Address: DR. REDDY''S LABORATORIES, INC., 200 SOMERSET CORPORATE BLVD, SEVENTH FLOOR, BRIDGEWATER, NJ, 08807-2862, US

(21) Appl. No.: 12/166,493

(22) Filed: Jul. 2, 2008

Related U.S. Application Data
(60) Provisional application No. 60/968,929, filed on Aug. 30, 2007.

(30) Foreign Application Priority Data
Jul. 2, 2007 (IN) 1412/CHE/2007

(51) Int. Cl. A61K 31/404; A61P 11/06

(52) U.S. Cl. 514/415

(57) ABSTRACT

Stable pharmaceutical compositions comprising zafirlukast and pharmaceutically acceptable salts, solvates, polymorphs, enantiomers or mixtures thereof, together with one or more pharmaceutically acceptable polymers, and processes for their preparation.

### INTRODUCTION

The present invention relates to stable pharmaceutical compositions comprising zafirlukast, including its pharmaceutically acceptable salts, solvates, polymorphs, enantiomers and mixtures thereof. More particularly the invention relates to stable compositions comprising zafirlukast with one or more pharmaceutically acceptable polymers and processes for their preparation. Further, the invention relates to solid oral pharmaceutical formulations comprising zafirlukast and processes for their preparation.

An aspect of the invention also relates to solubility-enhanced forms of zafirlukast or its salts.

The invention also relates to processes for preparing compositions comprising zafirlukast having a defined particle size range, and its pre-mixes.

The invention also relates to processes for preparing solubility-enhanced compositions comprising zafirlukast, and their methods of use.

Zafirlukast has a chemical name 4-(5-cyclopentyloxy-carbonylamino-1-methyl-indol-3-ylmethyl)-3-methoxy-N-o-tolylsulfonylbenzamide, and is structurally represented by Formula I.

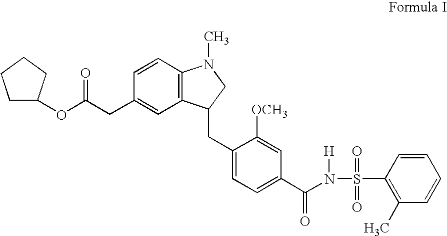

Zafirlukast is a selective and competitive receptor antagonist of leukotriene D4 and E4 (LTD4 and LTE4), components of slow-reacting substance of anaphylaxis (SRSA). Cysteinyl leukotriene production and receptor occupation have been correlated with the pathophysiology of asthma, including airway edema, smooth muscle constriction, and altered cellular activity associated with the inflammatory process, which contribute to the signs and symptoms of asthma.

The cysteinyl leukotrienes (LTC₄ LTD₄, LTE4) are the products of arachidonic acid metabolism and are various cells, including mast cells and eosinophills, these eicosinoids bind to cysteinyl leukotriene (CysLT) receptors. The CysLT type-1 (CysLT₁) receptor is found in human airway and other pro-inflammatory cells. CysLTs have been correlated with the pathophysiology of asthma.

Zafirlukast is a synthetic, selective peptide leukotriene receptor antagonist (LTRA), useful for the treatment of asthma and is commercially available in products sold under the brand name ACCOLATE™ as 10 and 20 mg tablets for oral administration. ACCOLATE™ is indicated for the prophylaxis and treatment of asthma in adults and children 5 years of age and older.

ACCOLATE™ film coated tablets contain amorphous zafirlukast as the active ingredient and the excipients croscarmellose sodium, lactose, magnesium stearate, microcrystalline cellulose, povidone, hypromellose, and titanium dioxide.

The greatest prevalence of asthma is in preschool children; however, the clinical utility of asthma therapy for this age group is limited by a narrow therapeutic index, long-term tolerability, and frequency and/or difficulty of administration. Asthma treatment requires an immediate perceivable effect. Inhalation therapy is a very common therapy prescribed for young children; inhalation therapy has the disadvantage of high dose variability.

Using a solid oral dosage form has the advantage of overcoming dose variability in comparison to inhaler therapy, which is very important in treating with a potent drug like zafirlukast for adults and pediatric patients aged 5 to 14 years. It is beneficial to use solubility-enhanced compositions of zafirlukast, where the zafirlukast has enhanced solubility so that the drug is quickly absorbed for immediate action.

U.S. Pat. No. 5,391,097 discloses zafirlukast, its pharmaceutical compositions, and processes for their preparation.

U.S. Pat. No. 5,482,963 discloses a pharmaceutical composition comprising amorphous zafirlukast and polyvinylpyrrolidone (PVP).

U.S. Pat. No. 5,612,367 discloses a method for stabilizing amorphous Form A of zafirlukast using PVP.

U.S. Pat. No. 5,993,859 discloses a process for the preparation of a zafirlukast co-precipitate with PVP by mixing zafirlukast, PVP, and water, and drying the mixture.

U.S. Pat. No. 6,143,775 discloses amorphous Form A of zafirlukast substantially free of other physical forms.

Other crystalline forms of zafirlukast and their compositions have been disclosed in U.S. Pat. Nos. 5,319,097 and 5,294,636.

The bioavailability of an orally administered drug, as measured by its entry into systemic circulation in the bloodstream, depends on at least two fundamental processes: drug dissolution in gastrointestinal fluids (in vivo drug release) and subsequent absorption of the dissolved drug. Several factors influence dissolution of a drug from its carrier, including surface area of the drug presented to the dissolution solvent medium, solubility of the drug substance in the solvent medium, and driving forces of the saturation concentration of dissolved materials in the solvent medium.

Zafirlukast exist in different polymorphs, wherein the polymorph with good bioavailability is unstable and stable polymorphs are with poor bioavailability. There is also reported that there is interconversion of one form into another. This poses a challenge to the formulation scientist to stabilize zafirlukast in the composition/formulation and also simultaneously improve the solubility of poorly soluble zafirlukast.

Towards this end, it has been the endeavor of pharmaceutical scientists to provide a stable solubility enhanced composition of zafirlukast, more specifically, thermodynamically stable with enhanced solubility, enhanced bioavailability and rapid onset of action.

There remains a need for soluble forms of zafirlukast that can be readily formulated for use in various modes of administration, including parenteral and oral administration.

### SUMMARY

The present invention relates to stable pharmaceutical compositions comprising zafirlukast, including its pharmaceutically acceptable salts, solvates, polymorphs, enantiomers or mixtures thereof. More particularly the invention relates to stable compositions comprising zafirlukast with one or more pharmaceutically acceptable polymers and processes of their preparation. Further, aspects of the invention relate to solid oral pharmaceutical formulations comprising zafirlukast and processes for their preparation.

An aspect of the present invention relates to solubility-enhanced pharmaceutical compositions comprising zafirlukast or pharmaceutically acceptable salts, solvates, polymorphs, enantiomers or mixtures thereof. More particularly, it relates to solubility-enhanced compositions comprising zafirlukast and one or more pharmaceutically acceptable polymers, and processes for their preparation.

An aspect of the present invention relates to stable compositions comprising zafirlukast or salts thereof.

In an embodiment the invention relates to stable compositions comprising zafirlukast or its salts and at least one pharmaceutically acceptable polymer.

In another embodiment the invention includes stable compositions comprising a solubility-enhanced form of zafirlukast.

Another aspect of the present invention relates to stable compositions comprising a solubility-enhanced form comprising a pre-mix containing zafirlukast or salts thereof.

In an embodiment the invention relates to stable compositions comprising zafirlukast and at least one pharmaceutically acceptable polymer.

In an embodiment the invention relates to stable compositions comprising zafirlukast and hydroxypropyl cellulose.

In an embodiment the invention relates to stable compositions comprising a pre-mix comprising zafirlukast and hydroxypropyl cellulose.

An aspect of the present invention relates to solubility-enhanced compositions comprising zafirlukast, wherein a premix comprises zafirlukast and a hydroxypropyl cellulose (HPC).

An aspect of the present invention relates to solubility-enhanced compositions comprising zafirlukast, where a premix comprises zafirlukast and a hydroxypropyl methylcellulose (HPMC).

An aspect of the present invention relates to solubility-enhanced pharmaceutical compositions comprising zafirlukast with a defined particle size distribution.

An aspect of the present invention relates to solubility-enhanced pharmaceutical compositions comprising zafirlukast with a defined particle size distribution of a pre-mix comprising zafirlukast or its salt.

In an embodiment the invention relates to stable compositions comprising zafirlukast or its pharmaceutically acceptable salts, where zafirlukast is combined with a cyclodextrin.

In an embodiment the invention relates to stable compositions comprising zafirlukast or its pharmaceutically acceptable salts, where zafirlukast is combined with a surfactant to enhance its solubility.

An aspect of the present invention relates to processes for preparing stable compositions comprising zafirlukast.

Another aspect of the present invention relates to processes for preparing stable compositions comprising solubility-enhanced forms of zafirlukast

In an embodiment the invention includes pharmaceutical formulations comprising compositions of zafirlukast.

In an embodiment the invention includes pharmaceutical formulations comprising solubility-enhanced forms of zafirlukast.

Yet another embodiment the invention relates to processes for preparing pharmaceutical formulations comprising zafirlukast or solubility-enhanced compositions of zafirlukast, and their methods of use.

An embodiment of the invention provides a pharmaceutical formulation comprising zafirlukast and a hydroxyalkyl cellulose, producing zafirlukast Cₘₐₓ values of about 280 ng/mL to about 440 ng/mL, AUC₀₋ₜ values of about 890 ng·hour/mL to about 1400 ng·hour/mL, and AUC_{0-α} values of about 910 ng·hour/mL to about 1425 ng·hour/mL, after oral administration of a single 20 mg zafirlukast dose to healthy humans.

### BRIEF DESCRIPTION OF THE DRAWINGS

FIG. 1 is an X-ray powder diffraction (XRPD) pattern of a stable composition comprising zafirlukast and HPC, prepared in Example 1.

FIG. 2 contains XRPD patterns of a stable composition comprising a zafirlukast HPC pre-mix prepared by direct compression, from Example 2.

FIG. 3 contains XRPD patterns of a stable composition comprising zafirlukast and HPC prepared by non-aqueous granulation, from Example 12.

FIG. 4 contains XRPD patterns of a stable composition comprising zafirlukast and HPC prepared by wet granulation, from Example 10.

FIG. 5 contains XRPD patterns of a stable composition comprising zafirlukast and HPMC, prepared in Example 11.

FIG. 6 is an XRPD pattern of a stable composition from Example 13B, after storage in a closed HDPE container at 40° C. and 75% RH for 3 months.

FIG. 7 is an XRPD pattern of a composition from Example 13B, prepared without any drug, after storage in a closed HDPE container at 40° C. and 75% RH for 3 months.

### DETAILED DESCRIPTION

The present invention relates to stable pharmaceutical compositions comprising zafirlukast, including its pharmaceutically acceptable salts, solvates, polymorphs, enantiomers and mixtures thereof. More particularly the invention relates to stable compositions of zafirlukast with one or more pharmaceutically acceptable polymers and processes of their preparation. Further, the invention relates to solid oral pharmaceutical formulations comprising zafirlukast and processes of their preparation.

An aspect of the invention relates to solubility enhanced forms of zafirlukast or its salts.

In an embodiment the invention relates to stable compositions comprising zafirlukast and at least one pharmaceutically acceptable polymer.

In an embodiment the invention relates to stable compositions comprising a solubility enhanced form of zafirlukast and at least one pharmaceutically acceptable polymer, wherein a solubility-enhanced form is a pre-mix composition.

In another embodiment the invention relates to stable compositions comprising zafirlukast and hydroxypropyl cellulose, hydroxyethyl cellulose, or hydroxypropyl methyl cellulose.

Zafirlukast is an oral leukotriene receptor antagonist (LTRA) for the maintenance treatment of asthma. Zafirlukast blocks the action of the cysteinyl leukotrienes on the CysLT1 receptors, thus reducing constriction of the airways, build-up of mucous in the lungs and inflammation of the breathing passages.

Various salts of zafirlukast that are useful in the invention include without limitation such as salts formed with bases which form a physiologically acceptable cation, such as alkali metals (e.g., sodium and potassium), alkaline earth metals (e.g., calcium and magnesium), aluminum, and ammonium salts, as well as salts made with appropriate organic bases such as triethylamine, morpholine, piperidine and triethanolamine. For the compound of Formula I which is sufficiently basic, examples of suitable pharmaceutically acceptable salts include acid-addition salts such as those made with a strong acid, for example hydrochloric, sulphuric, and phosphoric acids.

The term “zafirlukast” for purposes of the present invention includes any of its salts, polymorphs including any crystalline or amorphous forms, hydrates, solvates, enantiomers, etc.

The term “solubility enhanced” with respect to forms or compositions for purposes of the present invention relates to zafirlukast or its salts together with at least one pharmaceutically acceptable polymer, which may or may not be pre-mix compositions.

The term “pre-mix compositions” for purposes of the present invention relates to compositions comprising zafirlukast or its salts, wherein zafirlukast and at least one pharmaceutically acceptable polymer are in intimate contact with each other.

The term “stability” for purposes of the present invention relates to both stability with respect to physical form, such as a polymorphic form, and also chemical stability with respect to impurities.

The term “formulation” for purposes of the present invention relates to any composition comprising zafirlukast or its salts, a polymer and at least one pharmaceutically acceptable excipient.

Various parameters impacting compression processes include physical parameters of active ingredients or compositions, including compactability, flowability, moisture content (such as determined by Karl Fischer (KF) apparatus or infrared moisture balances), particle size (determined by sieve analyzer or Malvern particle size analyzer), bulk density and tapped density, compressibility index, Hausner ratio (determined by USP density apparatus), flow properties (determined by Flowdex apparatus), etc.

As zafirlukast is poorly soluble in water, particle size plays an important role in solubility. Particle size distribution is the percentage of particles with different sizes that exist in the total powder. It is represented in certain ways.

Particle size is the maximum dimension of a particle, normally expressed in micrometers. Particle size distributions are expressed in terms of, D₁₀, D₅₀, D₉₀ and D_{[4, 3]}, where D₁₀, D₅₀ and D₉₀ represent the 10^{th} percentile, the 50^{th} percentile and the 90^{th} percentile of the particle size distribution, respectively, as measured by volume. That is, the D₁₀, D₅₀, or D₉₀ is a value of the distribution such that 10%, 50%, and 90%, respectively, of the particles have a size of this value or less, or is the percentage of particles smaller than that size. D₅₀ is also known as median diameter of the particles. It is one of the important parameters representing characteristics of particle of powder. For a sample, if D₅₀=5 pm, it means 50% of the particles are smaller than 5 μm. Similarly D₁₀=5 μm, 10% particles are less than or equal to 5 μm, and D₉₀=5 μm, means that 90% of particles have sizes less than or equal to 5 μm. D_{[4,3]} represents the volume moment mean of the particle, or the volume weighted particle size.

Desired particle sizes of zafirlukast or pre-mixes can be achieved by various techniques known to the person skilled in the art, such as but not limited to being obtained directly from a synthesis process, or any particle size reduction processes can be used such as but not limited to sieving, sifting, milling, micronization, fluid energy milling, ball milling, and the like.

Particle size distribution is an indicator of how the drug substance is going to perform in terms of solubility, dissolution rate and bioavailability.

Therefore, in one embodiment the present invention provides a defined particle size distribution of zafirlukast or its salts, such that the zafirlukast or its salts have particle size distributions, wherein: D₉₀ of zafirlukast is less than about 200 μm or less than about 125 μm; D₅₀ of zafirlukast is less than about 50 μm, or less than about 30 μm, or less than about 10 μm; and D₁₀ of zafirlukast is less than about 10 μm, or less than about 5 μm.

In an embodiment the invention relates to particle size distributions of solubility-enhanced pharmaceutical compositions in the form of premixes, wherein: D₉₀ of a pre-mix comprising zafirlukast is less than about 200 μm, or less than about 120 μm, or less than about 50 μm; D₅₀ of a pre-mix comprising zafirlukast is less than about 50 μm, or less than about 30 μm; and D₁₀ of a pre-mix comprising zafirlukast is less than about 10 μm, or less than about 5 μm.

The compositions can be further characterized for physical parameters such as bulk density, tap density, moisture content, etc.

Bulk density is a property of particulate materials. It is the mass of a plurality of particles of the material, divided by the volume they occupy. The volume includes the space between particles as well as the space inside the pores of individual particles. Bulk density is not an intrinsic property of a material; it can change depending on how the material is handled. For example, particles poured into a cylinder will have a particular bulk density. If the cylinder is disturbed, the particles will move and settle closer together, resulting in a higher bulk density. For this reason, the bulk density of powders is usually reported both as “freely settled” and “tapped” density (where the tapped density refers to the bulk density of the powder after a specified compaction process, usually involving vibration of the container).

Physical mixtures of zafirlukast and excipients used for the preparation of pre-mixes can be made by mixing weighed quantities of zafirlukast and excipient used for the preparation of pre-mix, using suitable containers.

In embodiments, the invention relates to pharmaceutical compositions comprising zafirlukast or its salts, wherein tap densities of zafirlukast are in the range of about 0.6 g/ml to 0.9 g/ml, and bulk densities of zafirlukast are in the range of about 0.35 g/ml to 0.7 g/ml.

In embodiments, the invention relates to pharmaceutical compositions comprising solubilized form of zafirlukast or its salts wherein tap densities of a solubilized form of zafirlukast are less than about 0.85 g/ml, or less than about 0.75 g/ml, or less than about 0.65 g/ml and bulk densities are less than about 0.6 g/ml, or less than about 0.5 g/ml, or less than about 0.4 g/ml.

In an embodiment the invention relates to solubility-enhanced compositions of zafirlukast and at least one pharmaceutically acceptable polymer. In an embodiment of the present invention, solubility-enhanced compositions comprise pre-mixes comprising zafirlukast and pharmaceutical polymers, which have glass transition temperatures more than 150° C.

In an embodiment, the invention relates to solubility-enhanced compositions comprising zafirlukast or its salts and hydroxylalkyl cellulose.

In an embodiment, the invention relates to solubility-enhanced compositions comprising zafirlukast or its salts and hydroxypropyl cellulose as a polymer.

Further embodiments of the invention relate to solubility enhanced compositions comprising zafirlukast or its salts and cyclodextrin or its derivatives.

Zafirlukast exist in different polymorphic forms. Of the various polymorphs, a few are stable with poor bioavailability and a few are unstable with good bioavailability. The polymorphs are also prone to interconversion into other polymorphs under environmental conditions such as temperature, humidity, etc. pertaining to physical stability.

In embodiments, the present invention relates to stable compositions comprising zafirlukast together with at least one pharmaceutically acceptable polymer, characterized by their X-ray powder diffraction (XRPD) patterns, differential scanning calorimetry (DSC) curves, and nuclear magnetic resonance (NMR) spectra.

In an embodiment the invention relates to stable compositions and formulations comprising zafirlukast together with at least one polymer, wherein compositions and formulations during storage conditions, such as the accelerated stability testing conditions of 40° C. and 75% RH for about 3 months, retain the original XRPD patterns.

And also during the manufacturing process or during storage conditions, zafirlukast may be prone to various degradation reactions, resulting in the formation of impurities. Some of the impurities, which may be generated, are described below:

1) “ZAF-2” refers to 4-[5-cyclopentyloxycarbonyl)amino-1-methylindol-3-yl-methyl]-3-methoxy benzoic acid, represented by Formula II.

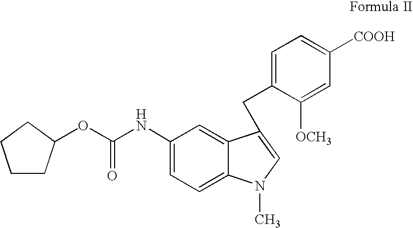

2) “ZAF-1” refers to methyl 4-[5-(cyclopentyloxycarbonyl)amino-1-methylindol-3-yl-methyl]-3-methoxy benzoate, represented by Formula III.

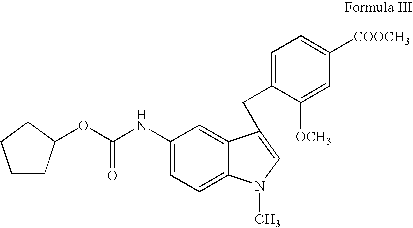

3) “ZAF-3(MCF)” refers to 3-methoxy-4-[(5-methoxycarbonyl)amino-1-methylindole-3-yl-methyl] benzoic acid 2-methyl benzene sulfonamide, represented by Formula IV.

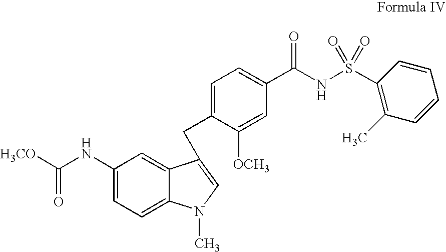

4) “ZAF-3(MTSA)” refers to [3-[[2-methoxy-4-[[[(3-methylphenyl)sulfonyl]amino]carbonyl]phenyl]methyl]-1-methyl-1H-indole-5-yl]carbamic acid cyclopentyl ester (meta isomer), represented by Formula V.

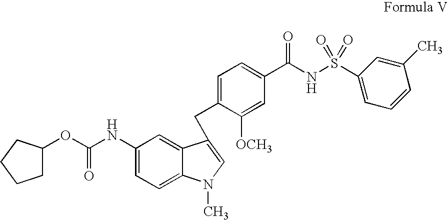

5) “ZAF (PTSA)” refers to [3-[[2-methoxy-4-[[[(4-methylphenyl)sulphonyl]aminocarbonyl]phenyl]methyl]-1-methyl-1H-indole-5-yl]carbamic acid cyclopentyl ester (para isomer), represented by Formula VI.

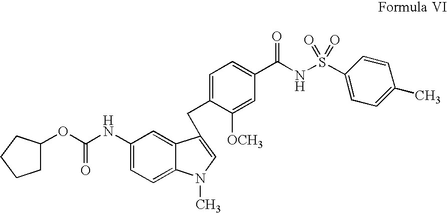

In an embodiment the invention includes stable compositions comprising zafirlukast or its salts.

In an embodiment the invention relates to stable compositions comprising zafirlukast or its salts and at least one pharmaceutically acceptable polymer.

In an embodiment the invention relates to stable compositions comprising zafirlukast or its salts and hydroxyalkyl celluloses.

In an embodiment the invention relates to stable compositions comprising zafirlukast or its salts and hydroxypropyl celluloses.

In embodiments, the invention provides compositions comprising zafirlukast or its pharmaceutically acceptable salt wherein the highest individual impurity is not more than about 0.5%, and the total impurities are not more than 1.5%, by weight of the zafirlukast content.

In an embodiment the invention includes stable compositions or formulations comprising zafirlukast or its salts wherein moisture contents of a composition or formulation is not more than about 8% w/w.

In embodiments of the present invention, pharmaceutically acceptable polymers for combining with zafirlukast include but are not limited to hydroxypropyl methylcelluloses, hydroxypropyl celluloses, hydroxypropyl methylcellulose acetate succinates, hydroxypropyl methylcellulose phthalates, other pharmaceutically acceptable polymers, and combinations thereof.

“Hydroxyalkyl cellulose,” as used herein, shall include cellulose derivatives that are substituted with a hydroxyalkyl group, wherein the alkyl group contains from about 1 to about 10 carbon atoms. Examples of suitable high-molecular weight hydroxyalkylcelluloses include, but are not limited to, hydroxymethylcelluloses, hydroxyethylcelluloses, hydroxypropylcelluloses, hydroxyethyl methylcelluloses, hydroxypropyl methylcelluloses, and the like. In one embodiment, the hydroxyalkylcellulose is a hydroxypropyl cellulose and/or hydroxypropyl methylcellulose.

Examples of suitable hydroxypropyl methylcelluloses (also called “hypromellose”) include those available with the designations HPMC K4M, HPMC K15M, and HPMC K100M. Examples of suitable hydroxypropyl celluloses include those available from Hercules, Inc. under the tradenames Klucel® H(CS) and Klucel® M.

In embodiments the present invention includes the use of hydroxypropyl cellulose (HPC) which is partially substituted poly(hydroxypropyl)ethers of cellulose. HPC is commercially available in a number of different grades, which have different solution viscosities. The molecular weight of the HPC ranges from about 50,000 to about 1,250,000. A useful HPC is available from Aqualon (a unit of Hercules Industrial Chemicals Pvt. Ltd., Mumbai, India) under the trademark KLUCEL. Suitable grades of HPC include: KLUCEL EF having a molecular weight of about 80,000; KLUCEL LF having a molecular weight of about 95,000; KLUCEL JF having a molecular weight of about 140,000; KLUCEL GF having a molecular weight of about 370,000; KLUCEL MF having a molecular weight of about 850,000; and KLUCEL HF is having a molecular weight of about 1,150,000. KLUCEL EXF has a viscosity of about 300 to 600 cps.

There are other suppliers of suitable HPC products, including Spectrum Chemical and Laboratory Products, California USA, Ruger Chemical Co. Ltd., New Jersey USA, and Biddle Sawyer Corporation, New York USA.

A stable composition of zafirlukast with HPC of the present invention may contain at least a detectable amount of zafirlukast present therein in amorphous form. It is believed that amorphous zafirlukast particles require less energy for dissolution than crystalline zafirlukast particles of similar dimensions, and that this reduced dissolution energy requirement contributes, at least in part, to an increased dissolution rate and decreased therapeutic onset time exhibited by amorphous zafirlukast and compositions thereof.

In an embodiment, zafirlukast compositions with pharmaceutically acceptable polymers comprising from about 10% to about 80%, or from about 15% to about 75%, or from about 25% to about 65%, by weight of polymer.

In another embodiment the invention relates to zafirlukast compositions with pharmaceutically acceptable polymers wherein weight ratios of zafirlukast to polymer are in the range of about 0.1:10 to 10:0.1, or about 0.1:5 to 5:0.1, or about 1:1 to about 5:1.

In embodiments, about 10% to 100%, or about 25% to 100%, or about 60% to 100%, by weight, of zafirlukast present in the composition is in the amorphous form.

Among the approaches that can be used to improve the solubility and dissolution properties of poorly soluble active ingredients, in order to overcome the problem of poor absorption, include forming inclusion complexes with cyclodextrins.

Further embodiments relates to stable compositions comprising zafirlukast or its salts and cyclodextrin or its derivatives.

Cyclodextrins with lipophilic inner cavities and hydrophilic outer surfaces are capable forming non-covalent inclusion complexes. The stability/solubility of the complex formed depends on how well the guest molecule fits into the cyclodextrin cavity. Without being bound by theory, it is felt that the processing of the zafirlukast along with the cyclodextrin provides a composition wherein the active is in intimate contact with the cyclodextrin, though not necessarily in the form of an inclusion complex. Thus, upon coming in contact with bio-relevant media, the active is forced into solution along with the cyclodextrin (in situ complex).

In one aspect of this embodiment zafirlukast and the cyclodextrin form a complex wherein complexation is complete or partial.

When the amount of the zafirlukast present is more than the amount which can be incorporated into an inclusion complex using the cyclodextrin selected, the remaining drug substance will be present in the form of a crystalline or amorphous drug substance as part of the mixture. Such solubilizing compositions are also within the scope of the invention without limitation. The amount of such free or uncomplexed drug present within the powder composition will be determined by the amount and type of the cyclodextrin, the complexation capacity of the cyclodextrin selected and the process utilized to prepare the powder composition and other parameters known to a person skilled in the art.

As used herein, “cyclodextrin” refers to any cyclodextrin which enhances the aqueous solubility and/or provides for effective delivery of a zafirlukast such as natural cyclodextrins, α-cyclodextrin, β-cyclodextrin, and γ-cyclodextrin, and their respective derivatives.

The natural cyclodextrins include α-cyclodextrin, β-cyclodextrin and γ-cyclodextrin; Derivatives are typically prepared by modifying the hydroxyl groups located on the exterior or hydrophilic side of the cyclodextrin. The modifications can be made to increase the aqueous solubility and the stability of the complex and can modify the physical characteristics of the complex including the formation and dissociation of the complex. The types and degree of modification, as well as their preparation, are well known in the art.

Derivatives of natural cyclodextrins such as alkylated cyclodextrins, comprising methyl-, dimethyl-, trimethyl- and ethyl-β-cyclodextrins; hydroxyalkylated cyclodextrins, including hydroxyethyl-, hydroxypropyl-, and dihydroxypropyl-β-cyclodextrin; ethylcarboxymethyl cyclodextrins; sulfate, sulfonate and sulfoalkyl cyclodextrins, such as β-cyclodextrin sulfate, β-cyclodextrin sulfonate, and β-cyclodextrin sulfobutyl ether; as well as polymeric cyclodextrins. Other cyclodextrin derivatives can be made by substitution of the hydroxy groups with saccharides, such as glucosyl- and maltosyl-β-cyclodextrin. methyl-β-cyclodextrin, dimethyl-β-cyclodextrin, trimethyl-β-cyclodextrin, 2-hydroxymethyl-β-cyclodextrin, hydroxyethyl-β-cyclodextrin, 2-hydroxypropyl-β-cyclodextrin, 3-hydroxypropyl-β-cyclodextrin, β-cyclodextrin sulfate, β-cyclodextrin sulfonate, or β-cyclodextrin sulfobutyl ether.

Any of the above cyclodextrins or their derivatives or polymers prepared from them can be used for preparation of the compositions of the invention, either alone or in the form of mixtures of one or more cyclodextrins.

Commercially available cyclodextrins may be used, such as those available from any of the commercial suppliers such as for example CARGILL, ROQUETTE, Aldrich Chemical Company, Milwaukee Wis. USA, and Wacker Chemicals, New Canaan, Conn. USA, or the cyclodextrins may be synthesized by any of the processes known in the art for the synthesis of cyclodextrins and their derivatives.

Various methods are known in the art to prepare drug-cyclodextrin complexes, including the solution method, co-precipitation method, the slurry method, the kneading method, and the grinding method. See T. Loftsson, Pharmaceutical Technology, 1999, 12, pages 41-50.

In the solution method, the drug, either as a solid or in a solution, is added to a solution containing an excess amount of cyclodextrin. It is also possible to add an excess of the drug to an aqueous cyclodextrin solution. The mixture is agitated, and may optionally be heated, until equilibrium is reached, which may take several hours or several days. The equilibrated solution is then filtered or centrifuged to give a clear solution of the drug-cyclodextrin complex. The clear solution can be directly administered to a subject, or a solid complex can be obtained by removal of the water by evaporation (such as spray-drying), sublimation (such as lyophilization) or other drying means well known in the art.

A solid complex may also be obtained by the precipitation method. Often, the cyclodextrin complexes will precipitate upon cooling of the solution. Otherwise, a solvent in which the complex has minimal solubility, typically an organic solvent, is used to precipitate the solid complex. The precipitate containing the complex can then be filtered or centrifuged to obtain a solid drug-cyclodextrin complex. A generally less effective method of preparing a solid complex mixture is to grind a dry mixture of the drug and cyclodextrin in a sealed container, which is then gently heated to a temperature between 60° C. and 140° C.

If the drug is poorly water-soluble, the slurry or kneading methods can be employed. The drug and cyclodextrin can be suspended in water to form slurry, which is similarly stirred and/or heated to equilibration. The complex can be collected by filtration or by evaporation of the water. The kneading method is similar to the slurry method, whereby the drug and cyclodextrin are mixed with a minimal amount of water to form a paste. The complex can be isolated by methods similar to those discussed above.

Suitable solvents which can be used for dissolving zafirlukast either alone or together with a pharmaceutically acceptable polymer include but are not limited to: alcohols such as methanol, ethanol, isopropyl alcohol, n-propanol, and the like; halogenated hydrocarbons such as dichloromethane, 1,2-dichloroethane, chloroform, carbon tetrachloride and the like; ketones such as acetone, ethyl methyl ketone, methyl isobutyl ketone and the like; esters such as ethyl acetate, n-propyl acetate, n-butyl acetate, t-butyl acetate and the like; ethers such as diethyl ether, dimethyl ether, diisopropyl ether, 1,4-dioxane and the like; hydrocarbons such as toluene, xylene, n-heptane, cyclohexane, n-hexane and the like; nitriles such as acetonitrile, propionitrile and the like; and mixtures thereof or their combinations with water in various proportions.

Another aspect of the present invention provides processes for the preparation of stable solubility-enhanced pre-mix compositions, wherein process comprises

a) providing a solution comprising zafirlukast and a polymer in a suitable solvent; and

b) removing solvent from the solution.

The pre-mix products so formed are intimate mixtures, in which individual particles of the individual ingredients are not distinguishable, such as using an optical microscope. Certain of the products are considered to have the nature of solid solutions, in which the initial components are dispersed on a molecular level.

The formulations of the present invention can be prepared using any processing operations, such as for example one or more of direct compression, dry granulation and wet granulation. Further, a wet granulation method may be conducted using either aqueous or non-aqueous solvents.

In a specific embodiment, a process for the preparation of formulation comprising zafirlukast or its solubility-enhanced composition comprises:

1) Sifting zafirlukast or its pre-mix and excipients such as diluents, disintegrants, binders, glidants, lubricants, etc. through a sieve;

2) dry mixing sifted ingredients;

3) optionally granulating the step 2) materials using binder/drug-binder/drug-polymer solution or dispersion, and subsequently drying and sizing through a sieve;

4) optionally compacting the step 2) materials into compacts and subsequently milling and sizing through a sieve;

5) placing either step 2) or step 3) or step 4) product into a suitable blender, adding sifted glidants and other excipients, if any, to the blender and blending;

6) adding sifted lubricant to step 5) materials and blending;

7) filling the step 6) product into capsules or compressing into tablets.

In an aspect, the present invention provides processes for the preparation of compositions, wherein zafirlukast and a polymer are in a solution, which is used to granulate or load onto mixtures of powdered excipients, beads, or particle mixtures.

In an embodiment the invention relates to methods of treating allergic disorders such as asthma, using pharmaceutical compositions of the present invention.

The processes of the present invention provide stable compositions of zafirlukast with pharmaceutically acceptable polymers; the term “stable composition of zafirlukast” refers to stability of the polymorphic form during storage at any commonly used temperature and humidity conditions for stability testing of pharmaceutical products, wherein the stability is evaluated by preservation of the original polymorphic form and purity in a composition.

Drying can be suitably carried out using equipment such as a tray dryer, vacuum oven, air oven, or using a fluidized bed drier, spin flash dryer, or flash dryer, under atmospheric or reduced pressures. The guideline residual solvent level depends on the type of solvent, but is not more than about 5000 ppm, or about 4000 ppm, or about 3000 ppm.

Other aspects of the present invention provide pharmaceutical formulations comprising stable compositions of zafirlukast with a polymer, and one or more pharmaceutically acceptable excipients.

The pharmaceutical compositions comprising a stable composition of zafirlukast with pharmaceutically acceptable polymer of the present invention may be formulated as: solid oral dosage forms such as, but not limited to, powders, granules, pellets, tablets, and capsules; liquid oral dosage forms such as but not limited to syrups, suspensions, dispersions, and emulsions; and injectable preparations such as but not limited to solutions, dispersions, and freeze dried compositions. Formulations may be in the form of immediate release, delayed release or modified release. Further, immediate release compositions may be conventional, dispersible, chewable, mouth dissolving, or flash melt preparations, and modified release compositions that may comprise hydrophilic or hydrophobic, or combinations of hydrophilic and hydrophobic, release rate controlling substances to form matrix or reservoir or combination of matrix and reservoir systems.

In embodiments of the present invention the compositions may be prepared using any of direct blending, dry granulation, wet granulation, or extrusion and spheronization steps. Compositions may be presented as uncoated, film coated, sugar coated, powder coated, enteric coated or modified release coated. Compositions of the present invention may further comprise one or more pharmaceutically acceptable excipients.

The solid dosage forms may include any number of excipients, such as, but not limited to, diluents, binding agents, disintegrants, coloring agents, lubricating agents, sweeteners, flavorings and flavor enhancing agents, taste-masking agents, preservatives, buffers, wetting agents, colorants or coloring agents, glidants and other excipients and combinations thereof. Desirably, the agents are chemically and physically compatible with the zafirlukast.

Pharmaceutically acceptable excipients that find use in the present invention include, but are not limited to:

### Diluents:

Various useful fillers or diluents include but are not limited to starches, lactose, mannitol (Pearlitol SD200), cellulose derivatives, confectioners sugar and the like. Different grades of lactose include but are not limited to lactose monohydrate, lactose DT (direct tableting), and lactose anhydrous, (Flowlac™ available from Meggle Products, Pharmatose™ available from DMV, and others). Different grades of starches include but are not limited to maize starch, potato starch, rice starch, wheat starch, pregelatinized starch (commercially available as PCS PC10 from Signet Chemical Corporation) and Starch 1500, Starch 1500 LM grade (low moisture content grade) from Colorcon, fully pregelatinized starch (commercially available as National 78-1551 from Essex Grain Products) and others. Different cellulose compounds that can be used include crystalline cellulose and powdered cellulose. Examples of crystalline cellulose products include but are not limited to CEOLUS™ KG801, Avicel™ PH101, PH102, PH301, PH302 and PH-F20, PH-112 microcrystalline cellulose 114, and microcrystalline cellulose 112. Other useful diluents include but are not limited to carmellose, sugar alcohols such as mannitol (Pearlitol™ SD200), sorbitol and xylitol, calcium carbonate, magnesium carbonate, dibasic calcium phosphate, and tribasic calcium phosphate.

### Disintegrants:

Various useful disintegrants include but are not limited to carmellose calcium (Gotoku Yakuhin Co., Ltd.), carboxymethylstarch sodium (Matsutani Kagaku Co., Ltd., Kimura Sangyo Co., Ltd., etc.), croscarmellose sodium(Ac-di-Sol™ FMC-Asahi Chemical Industry Co., Ltd.), crospovidone, examples of commercially available crospovidone products including but not limited to crosslinked povidone, Kollidon™ CL [manufactured by BASF (Germany)], Polyplasdone™ XL, XI-10, and INF-10 [manufactured by ISP Inc. (USA)], and low-substituted hydroxypropylcellulose. Examples of low-substituted hydroxypropylcellulose include but are not limited to low-substituted hydroxypropylcellulose LH11, LH21, LH31, LH22, LH32, LH20, LH30, LH32 and LH33 (all manufactured by Shin-Etsu Chemical Co., Ltd.). Other useful disintegrants include sodium starch glycolate, colloidal silicon dioxide, and starch.

### Glidants:

Glidants that are useful include colloidal silicon dioxide and the like; solubility or wetting enhancers such as anionic or cationic or neutral surfactants; complex forming agents such as various grades of cyclodextrins, resins; release rate controlling agents such as hydroxypropyl celluloses, hydroxyethyl celluloses, hydroxypropyl methylcelluloses, ethyl celluloses, methyl celluloses, various grades of methyl methacrylates, silicone dioxide, talc waxes and combinations thereof.

### Lubricants:

An effective amount of any generally accepted pharmaceutical tableting lubricant can be added to assist in compressing tablets. Useful tablet lubricants include magnesium stearate, glyceryl monostearates, palmitic acid, talc, carnauba wax, calcium stearate sodium, sodium or magnesium lauryl sulfate, calcium soaps, zinc stearate, polyoxyethylene monostearates, calcium silicate, silicon dioxide, hydrogenated vegetable oils and fats, stearic acid and combinations thereof.

Other pharmaceutically acceptable excipients that are of use include but are not limited to film formers, plasticizers, opacifiers, antiadhesives, polishing agents, colorants, flavoring agents, sweeteners, viscosity enhancers, preservatives, antioxidants and the like.

In addition to coating ingredients, sometimes commercially available pre-mixed coating materials such as Opadry™ White OY 58900 (contains hydroxypropyl methylcellulose, PEG 400, and titanium dioxide), Lusterclear™, etc. will be used. These typically require only mixing with a liquid before use.

The formulations that are prepared can be subjected to in vitro dissolution evaluation according to Test 711 “Dissolution” in United States Pharmacopoeia 29, United States Pharmacopeial Convention, Inc., Rockville, Md., 2005 (“USP”) to determine the rate at which the active substance is released from the dosage forms, and the content of active ingredient can conveniently be determined in solutions by techniques such as high performance liquid chromatography.

The formulations prepared are further packaged using appropriate packaging materials such as containers including lids, composed of polyethylene (high density polyethylene or low density polyethylene), and/or polypropylene, and/or glass, stainless steel bottles, etc. Also useful are various blisters or strips composed of aluminium or high-density polypropylene, or polyvinyl chloride, or polyvinyl chloride (PVC) coated with polyvinylidene dichloride (PVDC).

In the compositions of the present invention, zafirlukast is a useful active ingredient in the ranges of about 5 mg to about 30 mg, about 5 mg to about 25 mg, and about 10 mg to about 20 mg, per dosage unit.

Certain specific aspects and embodiments of this invention are described in further detail by the examples below, which examples are provided only for the purpose of illustration and are not intended to limit the scope of the appended claims in any manner.

### EXAMPLE 1

### Preparation of Zafirlukast Pre-Mix

Manufacturing Process:

a) 5 g of hydroxypropyl cellulose (Klucel™ LF) was dissolved in 75 ml of dichloromethane.

b) 5 g of zafirlukast was added to step a) solution and 130 ml of acetone was added.

c) Step b) mixture was stirred to get a clear solution.

d) The clear solution of step c) was spray-dried using an inlet temperature of 40-45° C.

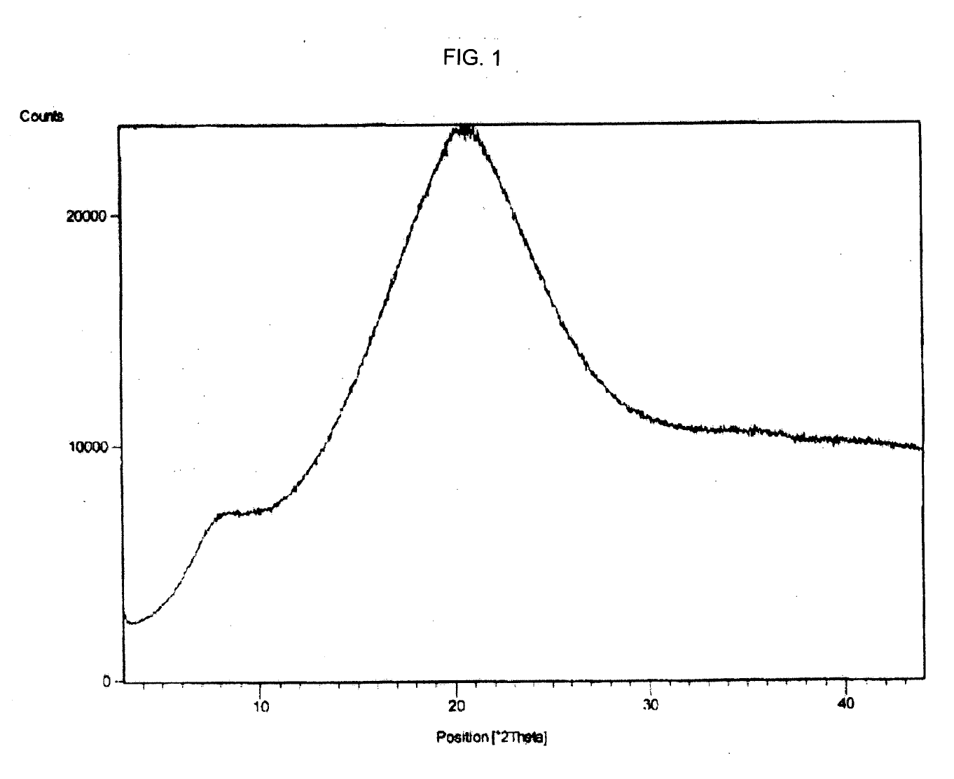

The spray-dried powder was analyzed by XRPD, giving the pattern of FIG. 1.

### EXAMPLE 2

### Formulations of Zafirlukast Pre-Mix

| Ingredient | mg/Tablet |
|---|---|
| Zafirlukast-HPC pre-mix (Example 1) | 40 |
| Microcrystalline cellulose (Avicel ™ PH 102) | 89.97 |
| Lactose (Flowlac ™ 100) | 58 |
| Croscarmellose sodium | 10 |
| Magnesium stearate | 2 |

Manufacturing Process:

a) Zafirlukast-hydroxypropyl cellulose pre-mix (1:1) and croscarmellose sodium were co-sifted through a ASTM #60 mesh sieve.

b) Lactose was added to materials of step a), sifted through a ASTM #40 mesh sieve, and mixed.

c) Microcrystalline cellulose was sifted through a ASTM #40 mesh sieve, added to materials of step b) and mixed in a double cone blender for 20 minutes.

d) Magnesium stearate was sifted through a ASTM #60 mesh sieve, added to materials of step c) and mixed well in a double cone blender for 5 minutes.

e) The lubricated blend of step d) was compressed into tablets using a compression machine.

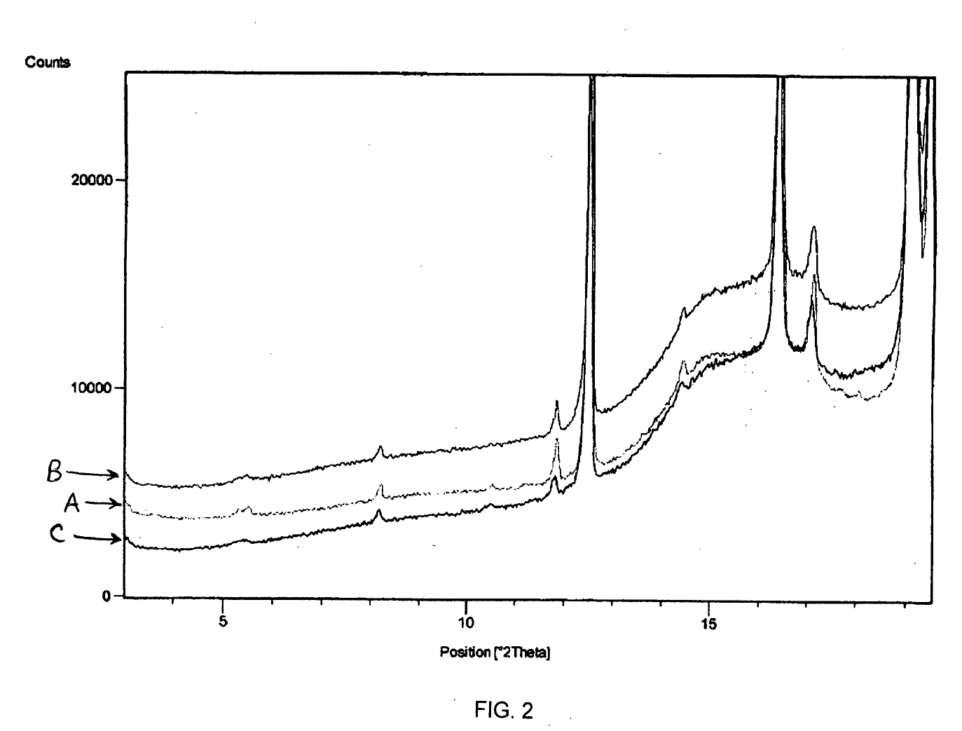

FIG. 2 is the XRPD pattern of the lubricated blend of step d). In the figure, “A” represents a blend that was prepared without any drug, “B” represents the blend, as prepared, and “C” represents the blend, after storage in a closed HDPE container at 40° C. and 75% RH for 3 months.

Physical characteristics of the step d) blend:

| Parameter | Value |
|---|---|
| Bulk density | 0.329 g/ml |
| Tapped density | 0.591 g/ml |

Particle size distribution

| | |
|---|---|
| D₉₀ | 25.01 μm |
| D₅₀ | 12.01 μm |
| D₁₀ | 3.82 μm |

Samples were stored at 40° C. and 75% relative humidity (“RH”) packaged in a closed HDPE container with a 1 gram silica gel desiccant pouch. Analyses were conducted before and after the storage, giving the following results, where values are percentages of the label zafirlukast content:

| | |
|---|---|
| Highest Single Impurity | Total Impurities |

| Initial | 3 months | Initial | 3 months |
|---|---|---|---|
| 0.086 | 0.083 | 0.27 | 0.23 |

XRPD analysis were done for samples of tablets, and the zafirlukast was in amorphous form after the samples were directly exposed for 4 weeks to 40° C. and 75% RH, and to 50° C.

### EXAMPLE 3

### Formulations of Zafirlukast Pre-Mix

| Ingredient | mg/Tablet |
|---|---|
| Zafirlukast-HPC pre-mix (Example 1) | 40 |
| Microcrystalline cellulose (Avicel PH 102) | 46 |
| Lactose (Flowlac 100) | 107 |
| Croscarmellose sodium | 6 |
| Magnesium stearate | 1 |

Manufacturing Process:

a) Zafirlukast-HPC pre-mix, croscarmellose sodium, microcrystalline cellulose, and lactose were sifted through a ASTM #40 mesh sieve and mixed in a double cone blender for 20 minutes.

b) Magnesium stearate was sifted through a ASTM #60 mesh sieve, added to materials from step a), and mixed well in a double cone blender for 5 minutes to form a lubricated blend.

c) The lubricated blend of step b) was compressed into tablets using a compression machine.

Physical characteristics of the blend of b):

| Parameter | Value | |
|---|---|---|
| Bulk density | 0.329 | g/ml |
| Tapped density | 0.591 | g/ml |

Particle size distribution

| | | |
|---|---|---|
| D₉₀ | 38.075 | μm |
| D₅₀ | 16.064 | μm |
| D₁₀ | 4.636 | μm |

### EXAMPLES 4-5

### Formulations of Zafirlukast Containing Zafirlukast Particles, by Direct Compression

| | Example 4 | Example 5 |
|---|---|---|
| Zafirlukast D₉₀ (μm) | 13.33 | 89.83 |
| Ingredient | mg/Tablet | |
| Zafirlukast | 20 | 20 |
| Microcrystalline cellulose (Avicel PH 102) | 42 | 42 |
| Lactose (Flowlac 100) | 127 | 127 |
| Croscarmellose sodium | 6 | 6 |
| Hydroxypropyl cellulose (Klucel ™ EXF) | 4 | 4 |
| Magnesium stearate | 1 | 1 |

Manufacturing Process:

a) Zafirlukast, croscarmellose sodium, microcrystalline cellulose, hydroxypropyl cellulose and lactose were sifted through a ASTM #40 mesh sieve and mixed in a double cone blender for 20 minutes.

b) Magnesium stearate was sifted through a ASTM #60 mesh sieve, added to materials of step a), and mixed well in a double cone blender for 5 minutes to form a lubricated blend.

c) The lubricated blend of step b) was compressed into tablets using a compression machine.

The above tablets of example 4 and 5 were tested for hardness, and for disintegration time by the USP test method, and results are in the following table.

| Tablet Characteristic | Example 4 | Example 5 |
|---|---|---|
| Hardness (Kp) | 8-10 | 8-10 |
| Disintegration Time (minutes) | 1-2 | 1-2 |

### EXAMPLES 6-11

### Formulations of Zafirlukast Containing Zafirlukast Particles, Prepared By Wet Granulation

| | | | | | Example | Example |
|---|---|---|---|---|---|---|
| | Example 6 | Example 7 | Example 8 | Example 9 | 10 | 11 |
| | Zafrilukast D₉₀ (μm) | | | | | |
| | 13.33 | 89.83 | 122.9 | 100.71 | — | — |
| Ingredient | mg/Tablet | | | | | |
| Zafirlukast | 20 | 20 | 20 | 20 | 20 | 20 |
| Lactose | 120 | 120 | 100 | 120 | 50 | 50 |
| monohydrate | | | | | | |
| Microcrystalline | 27 | 27 | 27 | 27 | 58 | 70 |
| cellulose | | | | | | |
| (Avicel PH 101) | | | | | | |
| Croscarmellose | 2 | 2 | 6 | 2 | 5 | 5 |
| sodium | | | | | | |
| Hydroxypropyl | 6 | 6 | 6 | 6 | 20 | — |
| cellulose | | | | | | |
| (Klucel LF) | | | | | | |
| Hypromellose 5 cps | — | — | — | — | — | 8 |
| Water‡ | 125 | 125 | 51 | 55 | 100 | 60 |
| Lactose | — | — | — | — | 20 | 20 |
| (Flowlac 100) | | | | | | |
| Microcrystalline | — | — | — | — | 58 | 70 |
| cellulose | | | | | | |
| (Avicel PH 101) | | | | | | |
| Microcrystalline | 20 | 20 | 35 | 20 | — | 20 |
| cellulose | | | | | | |
| (Avicel PH 102) | | | | | | |
| Croscarmellose | 4 | 4 | 4 | 4 | 5 | 5 |
| sodium | | | | | | |
| Magnesium | 1 | 1 | 2 | 1 | 2 | 2 |
| stearate | | | | | | |

‡Evaporates during processing.

Manufacturing Process:

a) Zafirlukast, lactose monohydrate, microcrystalline cellulose (first quantity) and croscarmellose sodium (first quantity) were sifted through a ASTM# 40 mesh sieve and loaded into a fluid bed processor.

b) Hydroxypropyl cellulose or hydroxypropyl methylcellulose was dissolved in water to form a binder solution.

c) The materials of step a) were granulated by spraying binder solution of step b) into the fluid bed processor and the granules were dried at 50-60° C. until the loss on drying was less than 3% w/w.

d) The dried granules were sifted through a ASTM # 30 mesh sieve.

e) Croscarmellose sodium and microcrystalline cellulose were sifted through a #40 mesh sieve, added to the step d) blend and mixed well in a double cone blender for 20 minutes

f) Magnesium stearate was sifted through a ASTM# 60 mesh sieve, added to materials of step e) and blended in a double cone blender for 5 minutes to form a lubricated blend.

g) The lubricated blend of step f) was compressed into tablets using a compression machine, giving the tablet characteristics below.

| Tablet Characteristic | Example 6 | Example 7 | Example 8 | Example 9 |
|---|---|---|---|---|
| Hardness (Kp) | 7-10 | 7-10 | 6-8 | 8-13 |
| Disintegration Time | 2-3 | 2-3 | 1-2 | 1-2 |
| (minutes) | | | | |

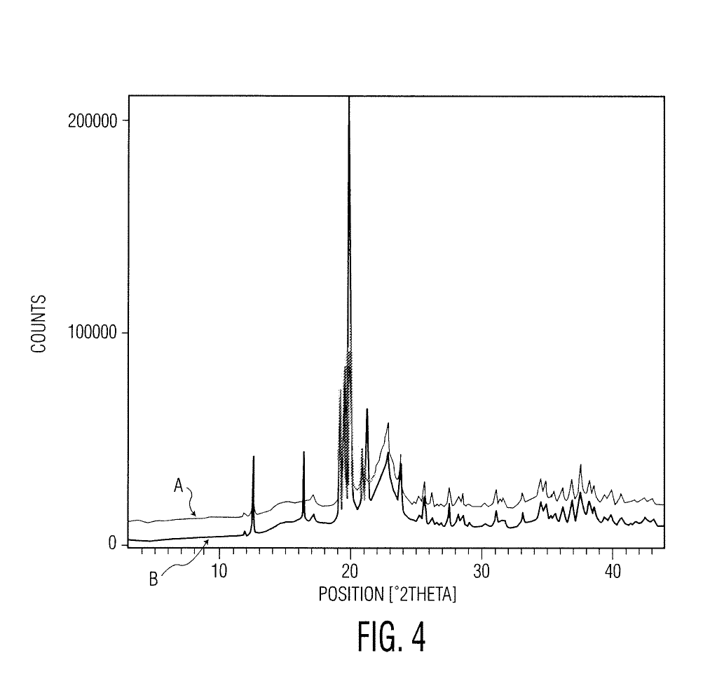

For Example 10, FIG. 4 is the XRPD pattern of the lubricated blend of step f). In the figure, “A” represents the blend, prepared without any drug, and “B” represents the blend prepared with drug. Hardness of the tablet: 7-10 Kp.

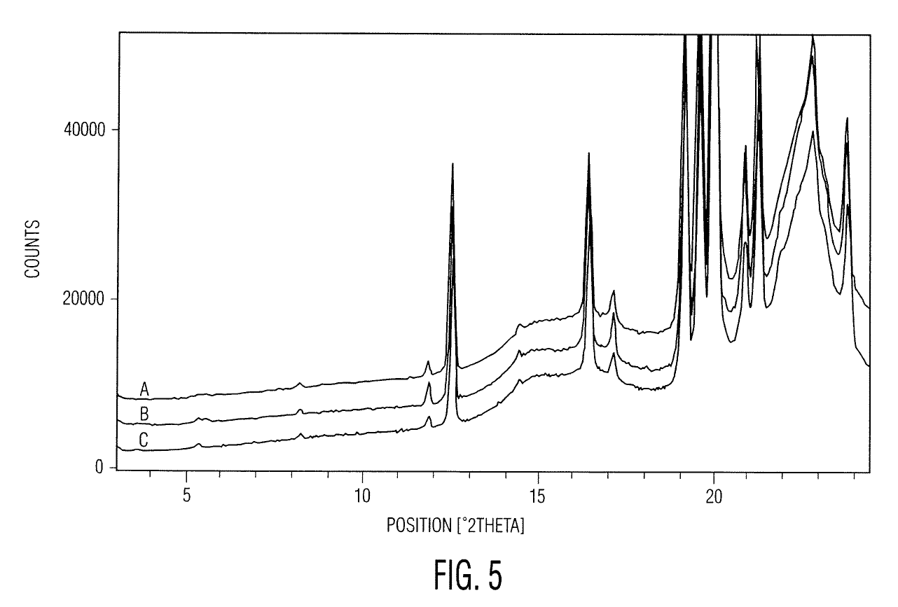

For Example 11, FIG. 5 is the XRPD pattern of the lubricated blend of step f). In the figure, “A” represents the blend, as prepared, “B” represents a blend, as prepared without any drug, and “C” represents the blend of “A” after storage in a closed HDPE container for 3 months at 40° C. and 75% RH. Hardness of the tablet: 4-7 Kp.

### EXAMPLE 12

### Formulations of Zafirlukast Containing Zafirlukast Particles, Prepared by Non-Aqueous Granulation

| Ingredient | mg/Tablet |
|---|---|
| Zafirlukast | 20 |
| Croscarmellose sodium | 5 |
| Hydroxypropyl cellulose (Klucel LF) | 40 |
| Microcrystalline cellulose (Avicel PH 101) | 40 |
| Lactose monohydrate | 70 |
| Acetone‡ | 500 |
| Methylene chloride‡ | 300 |
| Croscarmellose sodium | 10 |
| Microcrystalline cellulose (Avicel PH 102) | 23 |
| Magnesium stearate | 2 |

‡Evaporates during processing.

Manufacturing Process:

a) Acetone and methylene chloride were weighed into a beaker to form a solvent mixture.

b) Zafirlukast and Klucel were dissolved in the solvent of step a).

c) Croscarmellose sodium (first quantity), microcrystalline cellulose (PH 101) and lactose were sifted through a ASTM #60 mesh sieve, mixed in a double cone blender for 20 minutes and loaded into a fluid bed processor.

d) The solution of step b) was sprayed over the dry blend of step c) in the fluid bed processor at an inlet temperature of 55-65° C. to form granules, and the granules were dried at 55-65° C. for 60 minutes.

e) Croscarmellose sodium (second quantity) and microcrystalline cellulose (PH 102) were sifted through a ASTM #40 mesh sieve, added to the dried granules of step d) and blended in a double cone blender.

f) Magnesium stearate was sifted through a ASTM #60 mesh sieve, added to materials of step e) and blended to form a lubricated blend.

g) The lubricated blend of step f) was compressed into tablets using a compression machine.

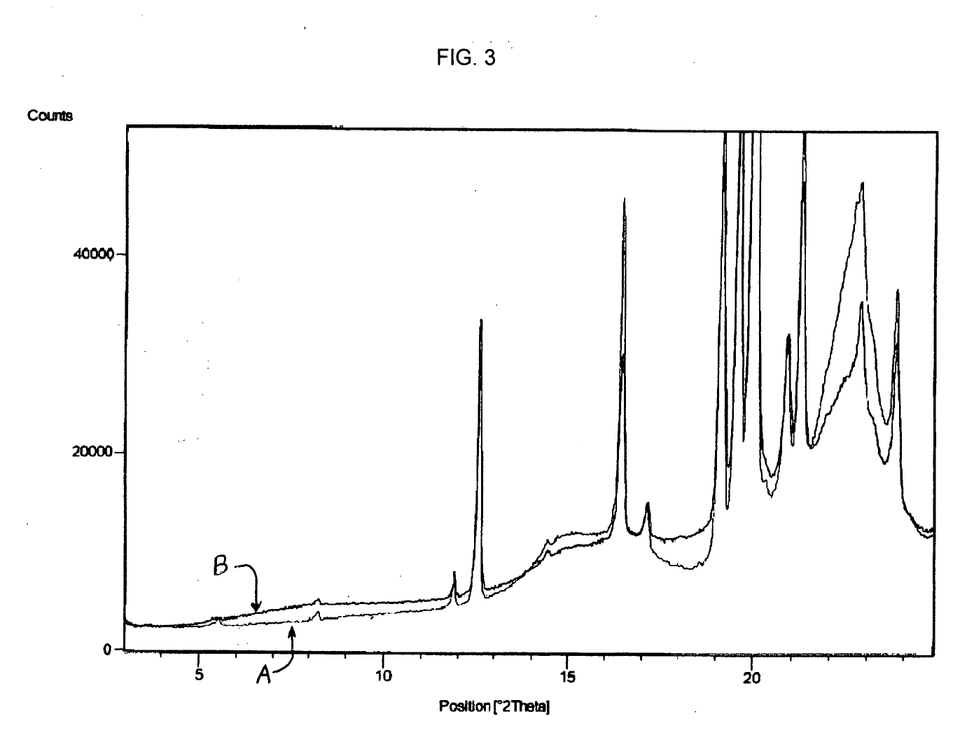

FIG. 3 is the XRPD pattern of the lubricated blend of step f). In the figure, “A” represents the blend, prepared without any drug, and “B” represents the blend prepared with drug, after storage in a closed HDPE container at 40° C. and 75% RH for 3 months.

Tablet hardness: 9-14 Kp.

In a storage stability test, six tablets were placed in a Petri dish and exposed to 40° C. and 75% RH conditions for 3 months. Impurities were analyzed by HPLC, and the values below are expressed as percentages of the label zafirlukast content.

| | |
|---|---|
| Maximum Single Impurity | Total Impurities |

| Initial | 3 Months | Initial | 3 Months |
|---|---|---|---|
| 0.083 | 0.081 | 0.27 | 0.22 |

### EXAMPLE 13

### Formulations of Zafirlukast Containing Zafirlukast Particles, Prepared by Wet Granulation

mg/Tablet

| Ingredient | 13A | 13B |
|---|---|---|
| Zafirlukast (amorphous) | 10 | 20 |
| Lactose monohydrate ** | 59 | 118 |
| Microcrystalline cellulose (Avicel PH 101) | 13.5 | 27 |
| Sodium starch glycolate Type A | 1.5 | 3 |
| Hydroxypropyl cellulose (Klucel LF) | 3 | 6 |
| Water‡ | 63.75 | 127.5 |
| Microcrystalline cellulose (Avicel PH 101) | 10 | 20 |
| Sodium starch glycolate Type A | 2 | 4 |
| Magnesium stearate | 1 | 2 |
| Opadry White OY-58900 | 3 | 6 |
| Water‡ | 32.5 | 65 |

‡Evaporates during processing.

Manufacturing Process:

1) Zafirlukast, having a particle size distribution with D₉₀=42 μm, and half of the lactose monohydrate were sifted through a ASTM #40 mesh sieve.

2) The mixture from step 1), the second half of the lactose monohydrate, microcrystalline cellulose (first quantity), and half of the sodium starch glycolate Type A were sifted through a ASTM #40 mesh sieve.

3) The step 2) mixture was sifted through a ASTM #40 mesh sieve.

4) Hydroxypropyl cellulose was added to water (first quantity) under constant stirring and stirred until a clear solution was formed.

5) Step 3) materials were loaded into a fluid bed processor and granulated using binder solution from step 4), and the granules obtained were dried until the loss on drying was between 0.5 and 2% w/w.

6) Dried granules from step 5) were sifted through ASTM #30 mesh sieve and the material retained on the sieve was collected.

7) Retained material from step 6) was milled in a comminuting mill fitted with a 1 mm screen at medium speed, knives forward. Milled granules were sifted through a ASTM #30 mesh sieve.

8) The granules that passed through a ASTM #30 mesh sieve from both of steps 6) and 7) were mixed together.

9) Microcrystalline cellulose (second quantity) and the second half of the sodium starch glycolate Type A were sifted through a ASTM #40 mesh sieve and magnesium stearate was sifted through a ASTM #60 mesh sieve.

10) Granules from step 8) and sifted microcrystalline cellulose and sodium starch glycolate from step 9) were loaded into a double cone blender and blended for about 20 minutes.

11) The blend of step 10) was blended with magnesium stearate of step 9) for about 5 minutes.

12) The lubricated blend of step 11) was compressed into tablets using compressing tooling.

13) Opadry white OY-58900 was added to water (second quantity) and mixed with a stirrer for 45 minutes until it formed a uniform suspension.

14) The tablets of step 12) were coated with coating suspension of step 13), and dried.

The coated tablets of examples 13A and 13B, and commercial ACCOLATE™ 20 mg tablets, were packaged in closed HDPE (high-density polyethylene) bottles and stored at 40° C. and 75% RH for 3 months. Impurity analytical results are tabulated below, where values are expressed as percentages of the label zafirlukast content.

| | | | ACCOLATE ™ | | | |
|---|---|---|---|---|---|---|
| | Example 13A | | 20 mg | | Example 13B | |
| Impurity | Initial | 3 Months | Initial | 3 Months | Initial | 3 Months |
| ZAF-1 | 0.05 | 0.07 | — | — | — | — |
| ZAF-3(MCF) | 0.08 | 0.08 | 0.04 | 0.04 | 0.08 | 0.06 |
| ZAF-3(MTSA) | 0.12 | 0.1 | 0.005 | 0.02 | 0.12 | 0.08 |
| ZAF(PTSA) | 0.02 | 0.01 | 0.02 | 0.01 | 0.02 | 0.02 |
| Highest | 0.06 | 0.06 | 0.05 | 0.04 | 0.06 | 0.05 |
| Unidentified | | | | | | |
| Total | 0.45 | 0.37 | 0.33 | 0.34 | 0.42 | 0.31 |
| Impurities | | | | | | |

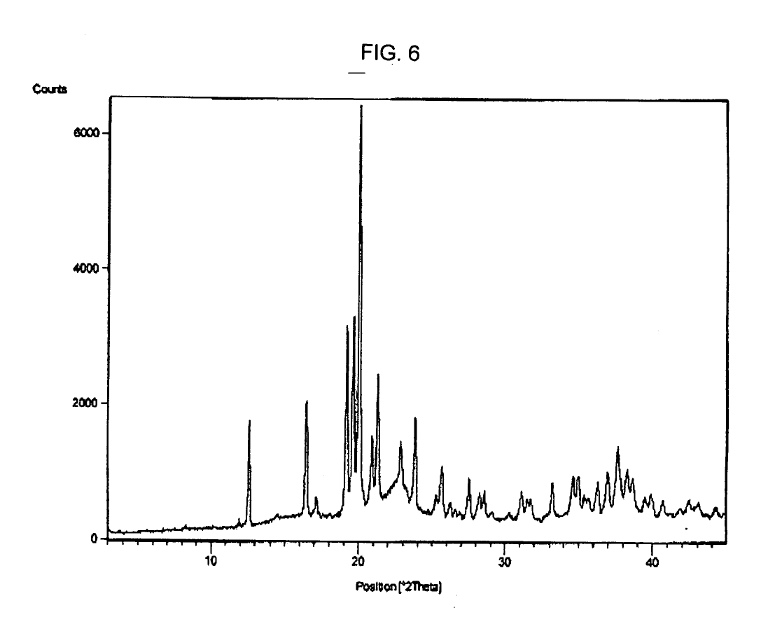

FIG. 6 is an XRPD pattern of a stable composition comprising zafirlukast prepared in Example 13B, after storage in a closed HDPE container at 40° C. and 75% RH for 3 months.

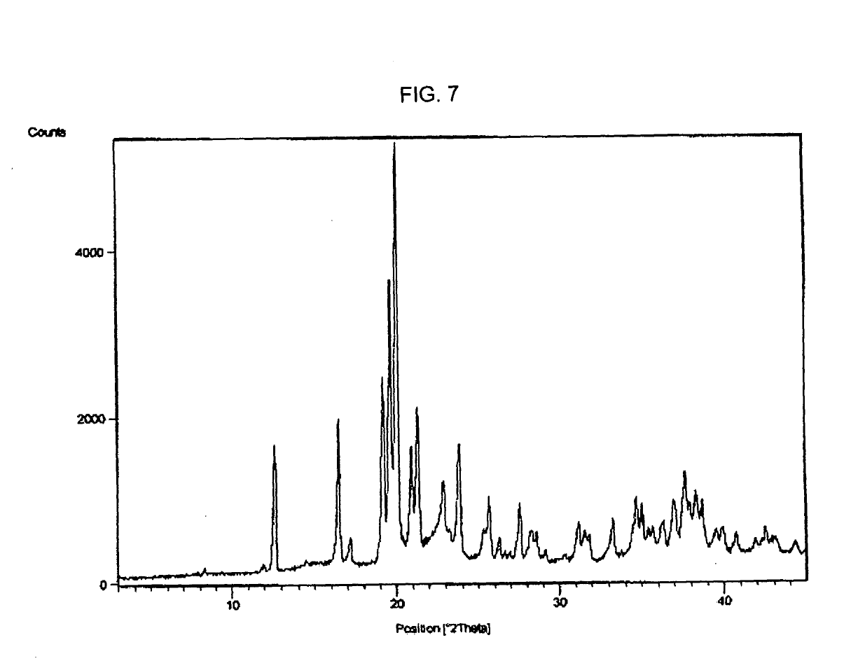

FIG. 7 is an XRPD pattern of a placebo composition for Example 13B (without any zafirlukast) after storage in a closed HDPE container at 40° C. and 75% RH for 3 months.

### EXAMPLE 14

### Comparative Dissolution Testing for Zafirlukast Compositions

Procedure used was according to Test 711 “Dissolution” in United States Pharmacopeia 29, United States Pharmacopeia Convention, Inc., Rockville, Md., 2005.

Dissolution conditions: 900 ml purified water with 0.5% sodium lauryl sulfate, 50 rpm stirring, and USP Type II apparatus.

Drug concentrations in solution were determined by high performance liquid chromatography.

The following table shows cumulative percentages of drug dissolved for zafirlukast 20 mg tablets and a commercial product.

| | Time (minutes) | | | | |
|---|---|---|---|---|---|
| | 10 | 20 | 30 | 45 | 60 |
| ACCOLATE ™ | 73 | 86 | 88 | 89 | 90 |
| 20 mg | | | | | |
| Example 2 | 70 | 76 | 79 | 81 | 81 |
| Example 3 | 70 | 81 | 84 | 87 | — |
| Example 4 | 69 | 79 | 83 | 84 | 87 |
| Example 6 | 66 | 82 | 85 | 88 | 84 |
| Example 9 | 54 | 73 | 81 | 86 | 90 |
| Example 13B | 92 | 98 | 99 | 99 | 99 |

The following table shows cumulative percentages of drug dissolved for zafirlukast 10 mg tablets and a commercial product.

| Time (minutes) | ACCOLATE ™ 10 mg | Example 13A |
|---|---|---|
| 10 | 82 | 63 |
| 20 | 89 | 91 |
| 30 | 90 | 94 |
| 45 | 91 | 95 |
| 60 | 92 | 95 |

### EXAMPLE 15

### Pharmacokinetic Study for the Composition of Example 13B

Tablets were evaluated in an open label, randomized two-period, two-treatment, two-sequence, single-dose, crossover, balanced fasting, study design involving administration of the test product and the commercial product ACCOLATE™ 20 mg in (fasting) healthy human volunteers, and plasma concentrations were determined at intervals after dosing.

The following parameters were calculated:

AUC₀₋ₜ= the area under plasma concentration versus time curve, from time zero to the last measurable concentration.

AUC_{0-∞}= area under the plasma concentration versus time curve, from time zero to infinity.

Cₘₐₓ= maximum plasma concentration.

The pharmacokinetic parameters from the fasting study were calculated and are summarized in the following table.

| | | | | | Ratio |
|---|---|---|---|---|---|
| | Example | | ACCOLATE ™ | | (100 × |
| Parameter | 13B (“T”) | | 20 mg (“R”) | | T ÷ R) |
| AUC₀₋ₜ | 1118.28 | ng · hour/mL | 1105.984 | ng · hour/mL | 101.11 |
| AUC_{0-∞} | 1138.999 | ng · hour/mL | 1127.469 | ng · hour/mL | 101.02 |
| Cₘₐₓ | 353.764 | ng/mL | 337.104 | ng/mL | 104.94 |

### EXAMPLE 16

### Pharmaceutical Formulation Comprising Zafirlukast with Cyclodextrin

| Ingredient | Quantity | |
|---|---|---|
| Zafrilukast | 1 | g |
| β-cyclodextrin (β-CD) | 1 | g |
| Microcrystalline cellulose | 11.8 | g |
| (MCC PH 102) | | |
| Croscarmellose sodium | 0.75 | g |
| (CCS) | | |
| Talc | 0.15 | g |
| Colloidal silicon dioxide | 0.15 | g |
| Magnesium stearate | 0.15 | g |
| Isopropyl alcohol (IPA)‡ | 14.5 | ml |
| Dicholoromethane (DCM)‡ | 16.5 | mL |

‡Evaporates during processing.

Manufacturing Process:

1) Dissolve zafirlukast and β-CD in the IPA-DCM mixture.

2) Granulate MCC PH 102 with the above-prepared solution and dry the granules.

3) Blend the dried granules with CCS.

4) Add talc and colloidal silicon dioxide (sifted through a ASTM #40 mesh sieve) and magnesium stearate (sifted through a ASTM # 80 mesh sieve), and mix thoroughly.

5) Compress the blend into tablets.

### EXAMPLE 17

### Pharmaceutical Formulation Comprising Zafirlukast with Cyclodextrin

| Ingredient | mg/Tablet |
|---|---|
| Zafirlukast | 20 |
| Hydroxypropyl-β-cyclodextrin (HPβCD) | 6 |
| Lactose monohydrate ** | 118 |
| Microcrystalline cellulose (Avicel PH 101) | 27 |
| Sodium starch glycolate Type A | 3 |
| Water‡ | 127.5 |
| Microcrystalline cellulose (Avicel PH 101) | 20 |
| Sodium starch glycolate Type A | 4 |
| Magnesium stearate | 2 |
| Opadry White OY-58900 | 6 |
| Water‡ | 65 |

‡Evaporates during processing.

Manufacturing Process:

1) Sift zafirlukast, lactose monohydrate through a ASTM #40 mesh sieve.

2) Sift microcrystalline cellulose (first quantity) and sodium starch glycolate Type A (first quantity) through a ASTM #40 mesh sieve.

3) Sift the step 2) mixture through a ASTM #40 mesh sieve.

4) Add HPβCD to water (first quantity) under constant stirring.

5) Granulate step 3) materials using cyclodextrin solution from step 4), and dry the granules obtained.

6) Sift the dried granules through a ASTM #30 mesh sieve.

7) Sift microcrystalline cellulose (second quantity) and sodium starch glycolate Type A (second quantity) through a ASTM #40 mesh sieve, and sift magnesium stearate through a ASTM #60 mesh sieve.

8) Blend granules from step 6) and microcrystalline cellulose and sodium starch glycolate from step 7) for about 20 minutes.

9) Mix the blend of step 8) with magnesium stearate of step 9) for about 5 minutes.

10) Compress the lubricated blend of step 9) into tablets using compression tooling.

11) Add Opadry white OY-58900 to water (second quantity) and mix with a stirrer for 45 minutes until it forms a uniform suspension.

12) Coat the tablets of step 12) with coating suspension of step 11), and dry.

We claim:

1. A solubility-enhanced form of zafirlukast, comprising zafirlukast and a hydroxyalkyl cellulose or a cyclodextrin.

2. The solubility-enhanced form of claim 1, wherein zafirlukast and a hydroxyalkyl cellulose are combined as a pre-mix.

3. The solubility-enhanced form of claim 1, wherein a solid comprising zafirlukast is granulated with a liquid comprising a hydroxyalkyl cellulose.

4. The solubility-enhanced form of claim 1, wherein a solid comprising a hydroxyalkyl cellulose is granulated with a liquid comprising zafirlukast.

5. The solubility-enhanced form of claim 1, wherein a solid comprising a pharmaceutical excipient is granulated with a liquid comprising a hydroxyalkyl cellulose and zafirlukast.

6. The solubility-enhanced form of claim 1, wherein a hydroxyalkyl cellulose comprises a hydroxypropyl cellulose, hydroxyethyl cellulose, or hydroxypropyl methylcellulose.

7. The solubility-enhanced form of claim 1, wherein weight ratios of zafirlukast to a hydroxylalkyl cellulose are about 0.1:10 to 10:0.1.

8. The solubility-enhanced form of claim 1, wherein weight ratios of zafirlukast to a hydroxylalkyl cellulose are about 0.5:5 to 5:0.5.

9. The solubility-enhanced form of claim 1, wherein weight ratios of zafirlukast to a hydroxyalkyl cellulose is about 1:1 to about 5:1.

10. The solubility-enhanced form of claim 1, wherein zafirlukast is complexed with a cyclodextrin.

11. The solubility-enhanced form of claim 1, being particles having a particle size distribution where Dgo is less than about 200 μm.

12. A pharmaceutical formulation comprising a solubility-enhanced form of claim 1.

13. The pharmaceutical formulation of claim 12 wherein zafirlukast and a hydroxyalkyl cellulose are combined as a pre-mix.

14. The pharmaceutical formulation of claim 12 wherein zafirlukast, optionally with a pharmaceutical excipient, is granulated with a liquid comprising hydroxyalkyl cellulose.

15. The pharmaceutical formulation of claim 12 wherein a hydroxyalkyl cellulose, optionally with a pharmaceutical excipient, is granulated with a liquid comprising zafirlukast.

16. The pharmaceutical formulation of claim 12 wherein a pharmaceutical excipient is granulated with a liquid comprising a hydroxyalkyl cellulose and zafirlukast.

17. The pharmaceutical formulation of claim 12 wherein a hydroxyalkyl cellulose comprises a hydroxypropyl cellulose, hydroxypropyl methylcellulose, or hydroxyethyl cellulose.

18. The pharmaceutical formulation of claim 12 wherein weight ratios of zafirlukast to a hydroxylalkyl cellulose are about 0.1:5 to 5:0.1.

19. The pharmaceutical formulation of claim 12, wherein a weight ratio of zafirlukast to a hydroxypropyl cellulose is about 3.33:1

20. The pharmaceutical formulation of claim 12 wherein a hydroxyalkyl cellulose comprises hydroxypropyl cellulose.

21. A pharmaceutical formulation, containing a mixture comprising zafirlukast and at least one pharmaceutical excipient, granulated with a solution comprising a hydroxypropyl cellulose.

22. The pharmaceutical formulation of claim 21, wherein a hydroxyalkyl cellulose comprises a hydroxypropyl cellulose, hydroxypropyl methylcellulose, or hydroxyethyl cellulose.

23. The pharmaceutical formulation of claim 21, wherein weight ratios of zafirlukast to hydroxyalkyl cellulose are about 1:1 to about 5:1.

24. The pharmaceutical formulation of claim 21, wherein a hydroxyalkyl cellulose comprises a hydroxypropyl cellulose and weight ratios of zafirlukast to hydroxypropyl cellulose are about 1:1 to about 5:1.

25. A pharmaceutical formulation comprising zafirlukast and a hydroxyalkyl cellulose, producing zafirlukast Cₘₐₓ values of about 280 ng/mL to about 440 ng/mL, AUC₀₋ₜ values of about 890 ng·hour/mL to about 1400 ng·hour/mL, and AUC_{0-α}, values of about 910 ng·hour/mL to about 1425 ng·hour/mL, after oral administration of a single 20 mg zafirlukast dose to healthy humans.

26. The pharmaceutical formulation of claim 25, wherein a hydroxyalkyl cellulose comprises hydroxypropyl cellulose.
